SEQUENCE LISTING (3 sequences)

SEQ ID NO: 1 - DNA, 20 bp, synthetic construct
  source 1..20: mol_type = other DNA
gtcacacttacagttcacac

SEQ ID NO: 2 - DNA, 25 bp, synthetic construct
  source 1..25: mol_type = other DNA
tcaggtcacacttacagttcacact

SEQ ID NO: 3 - protein, 14 aa, synthetic construct
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 13: note = 6-aminohexanoic acid
  MOD_RES 14: note = beta-alanine
RXRRXRRXRRXRXX

(19) United States
(12) Patent Application Publication
(10) Pub. No.: US 2024/0409932 A1
(43) Pub. Date: Dec. 12, 2024

(54) CORRECTION OF SPLICING MUTATIONS CAUSING PRIMARY CILIARY DYSKINESIA USING OLIGONUCLEOTIDES

(71) Applicant: The University of North Carolina at Chapel Hill, Chapel Hill, NC (US)

(72) Inventors: Silvia Kreda, Mebane, NC (US); Yan Dang, Chapel Hill, NC (US)

(21) Appl. No.: 18/700,418

(22) PCT Filed: Oct. 13, 2022
(86) PCT No.: PCT/US2022/078005

§ 371 Date: Apr. 11, 2024

Related U.S. Application Data
(60) Provisional application No. 63/255,768, filed on Oct. 14, 2021.

(51) Int. Cl. C12N 15/113; A61P 11/00

(52) U.S. Cl. CPC C12N 15/113; A61P 11/00; C12N 2310/11; C12N 2310/321; C12N 2310/3513; C12N 2320/33; C12N 2320/34

(57) ABSTRACT

This invention relates to the finding that novel splice switching oligonucleotides can correct splicing mutations. Moreover, the invention relates to using the novel splice switching oligonucleotides to correct the c.1167+1262A→G mutation in a pre-mRNA produced from the human CCDC39 gene and methods of using the same for treatment of primary ciliary dyskinesia (PCD) in a subject.

### STATEMENT OF PRIORITY

This application claims the benefit, under 35 U.S.C. § 119 (e), of U.S. Provisional Application No. 63/255,768, filed Oct. 14, 2021, the entire contents of which is incorporated by reference herein in its entirety.

### STATEMENT OF GOVERNMENT SUPPORT

This invention was made with government support under Grant No. TR002692 awarded by the National Institutes of Health. The government has certain rights in the invention.

### FIELD OF THE INVENTION

This invention relates to oligonucleotides for delivery to a subject and methods of using the same for treatment of primary ciliary dyskinesia (PCD) in a subject.

### BACKGROUND OF THE INVENTION

Lung diseases are one of the largest health burdens and are a main cause of mortality and morbidity worldwide. Yet, non-communicable and chronic pulmonary problems are often treated symptomatically without addressing pathogenesis.

Primary ciliary dyskinesia (PCD) is an autosomal recessive disorder and is the result of different defects in at least 50 identified genes involved in cilia formation and activity characterized by abnormal cilia and flagella that are found in the linings of the airway, the reproductive system, and other organs and tissues. For example, mutations in CCDC39 account for less than 10% of PCD cases and is a major cause of PCD in patients with ciliary radial spoke defects.

PCD occurs in approximately 1 in 7,500 people (Hannah et al., Lancet Respir/Med. 10 (5): 459 (2022)). Symptoms are present as early as at birth, with breathing problems, and the affected individuals develop frequent respiratory tract infections beginning in early childhood. People with PCD also have year-round nasal congestion and chronic cough. Chronic respiratory tract infections can result in a condition called bronchiectasis, which damages the passages, called bronchi, and can cause life-threatening breathing problems. Some individuals with PCD also have infertility, recurrent ear infections, and abnormally placed organs within their chest and abdomen. Defective cilia cannot produce the force and movement needed to eliminate fluid, microbes, and particles from the lungs. The movement of cilia also helps establish the left-right axis during embryonic development and propel the sperm cells forward to the female egg cell.

There is currently no cure for PCD (Paff et al., Int. J. Mol. Sci. 22:9834 (2021)). Current standard of care includes aggressive measures to enhance clearance of mucus and with antibiotic therapy for bacterial infections of the airways. Routine immunizations are administered to prevent respiratory infections and other secondary complications. For some patients, lobectomy, lung transplantation, and sinus surgery are considered. Gene therapy has been studied to address the urgent need for new, more effective treatments of PCD, but typically rely on delivery methods that are not suitable for in vivo use. In point of fact, efficient delivery of nucleic acid-based therapeutics to extra-hepatic organs remains a major obstacle (e.g., delivery to the lungs).

### SUMMARY OF THE INVENTION

This invention is based on the finding that novel splice switching oligonucleotides can correct a splicing mutation in CCDC39 and thereby treat PCD.

First, splice switching oligonucleotides (SSOs) are utilized that can be, e.g., peptide-morpholino oligomer conjugates (PPMOs). These molecules have a broad tissue distribution and, when given systemically, the SSOs can produce oligonucleotide effects in extra-hepatic tissues. Second, oligonucleotide endosomal compounds (OECs) can also be used. These are small molecules, discovered through high throughput screening, which selectively release oligonucleotides from non-productive entrapment in endosomal compartments. Thus, OECs allow oligonucleotides to access the cytosol and nucleus providing substantial enhancement of pharmacological effects. Herein, we describe use of novel SSOs, with and without OECs, to efficiently correct a splicing defect in CCDC39 in PCD patient-derived primary airway epithelial cells comprising an important CCDC39 splicing mutation.

Accordingly, one aspect of the invention is a splice switching oligonucleotide for correcting the c.1167+1262A→G mutation in the pre-mRNA produced from the human CCDC39 gene, wherein the oligonucleotide specifically hybridizes to an mRNA produced from the mutated CCDC39 gene at a site within 100 nucleotides of the mutation, optionally within 25 nucleotides of the mutation, optionally comprising at least 10 consecutive nucleotides of SEQ ID NO:1 or SEQ ID NO:2, optionally comprising a sequence at least 90% identical to SEQ ID NO:1 or SEQ ID NO: 2, or optionally comprising a sequence identical to SEQ ID NO:1 or SEQ ID NO:2.

In some embodiments, the SSO has one or more modifications (e.g., 2′-O-methoxyethyl, phosphorodiamidate morpholino).

In some embodiments, the SSO is conjugated to a peptide, optionally a peptide that is at least 90% identical to RXRRXRRXRRXRXB (SEQ ID NO:3), wherein R is arginine, B is β-alanine, and X is 6-aminohexanoic acid.

One aspect of the invention is a pharmaceutical composition comprising the SSO in a pharmaceutically acceptable carrier, optionally including an oligonucleotide endosomal compound.

One aspect of the invention is a method for correcting a c.1167+1262A→G mutation in the pre-mRNA produced from the CCDC39 gene in a cell, optionally in a subject.

Another aspect of the invention is a method of treating or delaying the onset of PCD in a subject.

These and other aspects of the invention are set forth in more detail in the description of the invention below.

### BRIEF DESCRIPTION OF THE DRAWINGS

FIG. 1 is a schematic showing an engineered mini-gene consisting of the EGFP cDNA interrupted by Intron IX of CCDC39 (NG_029581.1) encoding the c.1167+1261A→G splicing mutation, which was synthesized and expressed in epithelial cells.

FIG. 2 is a gel showing the effects of administration of an SSO (1 μM) with the wild type sequence into HEK cells expressing the mini-gene with vehicle or OEC (UNC7938, 10 μM). Splicing correction was analyzed by RT-PCR at 4, 6, 24, or 48 hours post-treatment, using 2 sets of primers (F′ and R′). The PCR bands were sequenced and confirmed to be mutant (˜300 bp) and WT (˜200 bp). This shows that normal “splicing out” of intron IX increased with time after treatment.

FIG. 3 is a gel showing mRNA extracts from induced pluripotent stem cells (iPSCs) from a normal (WT) and a PCD patient analyzed by RT-PCR.

FIG. 4 is a gel showing PCD patient generated iPSCs treated with SSOs (1 μM) with and without OEC (UNC7938, 10 μM). Controls included iPSCs from a normal patient, untreated PCD cells, a PPMO with a scrambled PMO sequence and a non-targeted PS SSO. GAPDH was used as a loading control. The PPMO prototype corrected the splicing mutation in the patient cells; and OEC was not necessary in these cells to increase delivery efficiency of the PPMO.

FIG. 5 shows SSO/PPMOCCDC39 Dose Response in iPSC derived from a patient homozygous for the CCDC39 c.1167+1261A>G splicing defect.

### DETAILED DESCRIPTION OF THE INVENTION

The present invention relates to a novel SSO to directly correct splicing defects, specifically, the c.1167+1261A→G splicing mutation in CCDC39, thereby treating primary ciliary dyskinesia (PCD). Additionally, the invention may have potential therapeutic implications in various pulmonary and non-pulmonary diseases that involve splicing defects.

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety. In case of a conflict in terminology, the present specification is controlling.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.822 and established usage. See, e.g., Patent In User Manual, 99-102 (November 1990) (U.S. Patent and Trademark Office).

Except as otherwise indicated, standard methods known to those skilled in the art may be used for cloning genes, amplifying and detecting nucleic acids, and the like. Such techniques are known to those skilled in the art. See, e.g., Green et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide sequence of this invention, means a polynucleotide that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to correct splicing of a target mRNA by at least about 50% or more as compared to the correction achieved with a polynucleotide consisting of the recited sequence.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of PCD, reduction in nasal congestion and coughing, prevention of respiratory tract infections and/or ear infections, prevention of infertility, or increase in survival time). Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Prevent” or “preventing” or “prevention” refer to prevention or delay of the onset of the disorder and/or a decrease in the severity of the disorder in a subject relative to the severity that would develop in the absence of the methods of the invention. The prevention can be complete, e.g., the total absence of PCD in a subject. The prevention can also be partial, such that the occurrence or severity of PCD in a subject is less than that which would have occurred without the present invention.

As used herein, the terms “protein” and “polypeptide” are used interchangeably and encompass both peptides and proteins, unless indicated otherwise.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When an oligonucleotide is produced synthetically, less common bases, such as inosine, 5-methylcytosine, 6-methyladenine, hypoxanthine and others can also be used. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The splice switching oligonucleotide or chemically-modified splice switching oligonucleotide may be constructed using chemical synthesis and enzymatic ligation reactions by procedures known in the art. For example, a splice switching oligonucleotide or chemically-modified splice switching oligonucleotide may be chemically synthesized using naturally occurring nucleotides or various modified nucleotides designed to increase the biological stability of the molecules or to increase the physical stability of the duplex formed between the splice switching oligonucleotide or chemically-modified splice switching oligonucleotide and target nucleotide sequences, e.g., phosphorothioate derivatives and acridine substituted nucleotides can be used. Examples of modified nucleotides which can be used to generate the splice switching oligonucleotide or chemically-modified splice switching oligonucleotide include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the oligonucleotide can be produced using an expression vector into which a nucleic acid encoding the splice switching oligonucleotide has been cloned.

The splice switching oligonucleotide or chemically-modified splice switching oligonucleotide can further include nucleotide sequences wherein at least one, or all, of the internucleotide bridging phosphate residues are modified phosphates, such as methyl phosphonates, methyl phosphonothioates, phosphoromorpholidates, phosphoropiperazidates, phosphoramidates, and phosphorodiamidates, including wherein one or more nucleotides form a morpholino backbone. For example, every one or every other one of the internucleotide bridging phosphate residues can be modified as described. In another non-limiting example, the splice switching oligonucleotide or chemically-modified splice switching oligonucleotide is a nucleotide sequence in which at least one, or all, of the nucleotides are modified, e.g., to contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). In another example, one or more of the nucleotides may be a 2′-fluoro nucleotide, a 2′-O-methyl nucleotide, 2′-O-methoxyethyl (MOE), or a locked nucleic acid nucleotide. Other examples include 5′ constrained ethyl oligonucleotides and tricyclo-DNA oligonucleotides. For example, every one or every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989); Walder and Walder, Proc. Natl. Acad. Sci. USA 85:5011 (1988); incorporated by reference herein in their entireties for their teaching of methods of making polynucleotide molecules, including those containing modified nucleotide bases).

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

By the term “express” or “expression” of a polynucleotide coding sequence, it is meant that the sequence is transcribed, and optionally, translated.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, “complementary” polynucleotides are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48: 1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

### Splice Switching Oligonucleotides

The invention presents an alternative therapeutic approach by targeting CCDC39 at the transcriptional level. The invention consists of splice switching oligonucleotides that can complementarily bind CCDC39 pre-mRNA and modulate the activity of the spliceosome, inducing correction of the CCDC39 c.1167+1262A→G splicing mutation and production of full length wild-type CCDC39 mRNA. Traditionally, oligonucleotides have faced clinical limitations due to their lack of tissue specificity, rapid degradation within the body, and immune activation. However, the synthetic SSOs herein contain novel chemical modifications that confer drug-like properties, which will protect them from in vivo degradation.

Accordingly, one aspect of the invention relates to a splice switching oligonucleotide, wherein the nucleotide sequence is at least 80% complementary (e.g., at least 85%, 90%, 91%, 92%, 93%, 94%, 95%, 96%, 97%, 98%, or 99% complementary) to an intronic region of the CCDC39 gene, the region consisting essentially of about 15 to about 40 consecutive nucleotides, e.g., about 20 to about 35 consecutive nucleotides, about 20 to about 30 consecutive nucleotides, or any range therein; wherein the splice switching oligonucleotide corrects an intronic splicing mutation in the CCDC39 gene. The splice switching oligonucleotide provides increased expression of a wild-type CCDC39 protein in a cell as compared to cells without the splice switching oligonucleotide (e.g., a control cell, non-transfected cell, or a cell expressing mutant CCDC39 or minimal or no CCDC39). In some embodiments, expression of wild-type CCDC39 is at least about 5% compared to a normal cell (e.g., a cell without a CCDC39 mutation), e.g., at least about 5%, 10%, 20%, 30%, 40%, 50%, 60%, 70%, 80%, 90%, 95%, or more. The sequence of the human CCDC39 gene on which the oligonucleotide is based is known in the art and can be found, e.g., in Accession Nos. NG_029581.1 and NM_181426.2, incorporated herein by reference in their entirety. The CCDC39 c.1167+1262A→G splicing mutation is depicted in Accession No. rs577069249 in the SNP database (dbSNP), incorporated herein by reference in its entirety.

Additional nucleotides can be added at the 3′ end, the 5′ end, or both the 3′ and 5′ ends to facilitate manipulation of the splice switching oligonucleotide but that do not materially affect the basic characteristics or function of the splice switching oligonucleotide. Additionally, one or two nucleotides can be deleted from one or both ends of any of the sequences disclosed herein that do not materially affect the basic characteristics or function of the splice switching oligonucleotide. The term “materially affect” as used herein refers to a change in the ability to correct expression of the wild-type protein encoded by the mRNA by no more than about 50%, e.g., no more than about 50%, 45%, 40%, 35%, 30%, 25%, 20%, 15%, 10%, or less.

In particular embodiments, the present invention provides a splice switching oligonucleotide containing a nucleotide sequence that is fully complementary to a region of the target gene. However, it is to be understood that 100% complementarity between the splice switching oligonucleotide and the target sequence is not required to practice the present invention. Thus, sequence variations that might be expected due to genetic mutation, strain polymorphism, or evolutionary divergence can be tolerated. Oligonucleotides with insertions, deletions, and single point mutations relative to the target sequence may also be effective for inhibition.

In some embodiments, the nucleotide sequence of the splice switching oligonucleotide comprises at least 5 consecutive nucleotides of the sequence of SEQ ID NO:1 or SEQ ID NO:2 (e.g., at least 5, 6, 7, 8, 9, 10, 11, 12, 13, 14, 15, 16, 17, 18, 19, 20, or more consecutive nucleotides of the sequence of SEQ ID NO:1 or SEQ ID NO:2).

In some embodiments, the nucleotide sequence of the splice switching oligonucleotide comprises a nucleotide sequence that is at least about 70% identical to the nucleotide sequence of any of SEQ ID NOS: 1-2, e.g., at least about 70%, 75%, 80%, 85%, 90%, 91%, 92%, 93%, 94%, 95%, 96%, 97%, 98%, 99%, or more identical to the nucleotide sequence of any of SEQ ID NOS: 1-2. In some embodiments, the nucleotide sequence of the splice switching oligonucleotide comprises, consists essentially of, or consist of the nucleotide sequence of any of SEQ ID NOS: 1-2.

SEQ ID NO: 1
GTCACACTTACAGTTCACAC
SEQ ID NO: 2
TCAGGTCACACTTACAGTTCACACT

In particular embodiments, the present invention provides a splice switching oligonucleotide containing a nucleotide sequence that is fully complementary to an intronic region of the target gene for correcting a splicing mutation. However, it is to be understood that the splice switching oligonucleotide does not necessarily need to overlap the intronic region containing the mutation. In some embodiments, the splice switching oligonucleotide is complementary to a region of the gene up to 100 nucleotides (e.g., up to 100 nucleotides, 75 nucleotides, 50 nucleotides, 25 nucleotides, 20 nucleotides, 15 nucleotides, 10 nucleotides, or 5 nucleotides) upstream of the mutation. In some embodiments, the splice switching oligonucleotide is complementary to a region of the gene up to 100 nucleotides (e.g., up to 100 nucleotides, 75 nucleotides, 50 nucleotides, 25 nucleotides, 20 nucleotides, 15 nucleotides, 10 nucleotides, or 5 nucleotides) downstream of the mutation. Thus, complementary sequences do not necessarily need to overlap the mutation in order to practice the invention. A splice switching oligonucleotide not overlapping the mutation may also be effective for correcting the splicing mutation and expressing normal CCDC39.

In some embodiments, nuclear localization signals can be used to enhance the targeting of the splice switching oligonucleotide into the proximity of the nucleus and/or its entry into the nucleus. Such nuclear localization signals can be a protein or a peptide such as the SV40 large Tag NLS or the nucleoplasmin NLS. These nuclear localization signals interact with a variety of nuclear transport factors such as the NLS receptor (karyopherin alpha) which then interacts with karyopherin beta.

In some embodiments, the splice switching oligonucleotide is conjugated (e.g., through a morpholino group) to a peptide that enhances the delivery and/or activity of the oligonucleotide, e.g., a cationic peptide. In one embodiment, the peptide is at least 70% identical to RXRRXRRXRRXRXB (SEQ ID NO:3), e.g., at least 70%, 75%, 80%, 85%, 90%, 91%, 92%, 93%, 94%, 95%, 96%, 97%, 98%, 99%, or 100% identical to RXRRXRRXRRXRXB (SEQ ID NO: 3), wherein R is arginine, B is-alanine, and X is 6-aminohexanoic acid.

In some embodiments, one or more nucleotides of the splice switching oligonucleotide are chemically modified nucleotides and/or the backbone of the oligonucleotide is chemically modified. In some embodiments, one, all, or fewer than all the nucleotides are modified. In some embodiments, at least 1, 2, 3, 4, 5, 6, 7, 8, 9, 10, 11, 12, 13, 14, 15, 16, 17, 18, 19, or 20 or more nucleotides are modified. In some embodiments, the splice switching oligonucleotide has more than one type of modification. In some embodiments, the splice switching oligonucleotide comprises one or more phosphorodiamidate morpholino nucleotides, forming at least part of a morpholino backbone. In other embodiments, all the nucleotides of the splice switching oligonucleotide are phosphorodiamidate morpholino nucleotides, forming a morpholino backbone.

In some embodiments, the splice switching oligonucleotide comprises one or more 2′-O-methoxyethyl nucleotides (MOE). In other embodiments, all the nucleotides of the splice switching oligonucleotide are 2′-O-methoxyethyl nucleotides. The MOE-modified oligonucleotide may have additional modifications, e.g., a phosphorothioate backbone.

Delivery into Target Cells and/or Nucleus

As used herein, the terms “contacting,” “introducing” and “administering” are used interchangeably, and refer to a process by which SSOs of the present invention or a nucleic acid molecule encoding a SSO of this invention is delivered to a cell, in order to inhibit or alter or modify expression of a target gene. The SSO may be administered in a number of ways, including, but not limited to, direct introduction into a cell (i.e., intracellularly) and/or extracellular introduction into a cavity, interstitial space, or into the circulation of the organism, e.g., the nose, lung, ear, eye, or intestine.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transfection event or in separate transfection events. Thus, the term “transfection” as used herein refers to the introduction of a heterologous nucleic acid into a cell. Transfection of a cell may be stable or transient.

“Transient transfection” in the context of a polynucleotide means that a polynucleotide is introduced into the cell and does not integrate into the genome of the cell.

According to the present invention, a splice switching oligonucleotide as described herein may be delivered as naked nucleic acid (unpackaged) or via delivery vehicles. As used herein, the terms “delivery vehicle,” “transfer vehicle,” “nanoparticle” or grammatical equivalent, are used interchangeably.

In some embodiments, a splice switching oligonucleotide may be delivered via a single delivery vehicle. In some embodiments, a splice switching oligonucleotide may be delivered via one or more delivery vehicles each of a different composition. According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags, and vehicles suitable for e.g., intravenous, subcutaneous, intramuscular, intranasal, intrapulmonary, ocular, otic, or intrathecal delivery.

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as lung, pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931) and/or peptides as described above.

In particular embodiments, a splice switching oligonucleotide according to the present invention is administered to the subject to treat, delay the onset of and/or prevent PCD.

Thus, as one aspect, the invention further encompasses a method of delivering a splice switching oligonucleotide to a subject, comprising administering to the subject an effective amount of the splice switching oligonucleotide, thereby delivering the splice switching oligonucleotide to the subject.

In another aspect, the invention further encompasses a method of treating, delaying the onset of, and/or preventing PCD in a subject in need thereof, comprising administering to the subject a therapeutically effective amount of a splice switching oligonucleotide, thereby treating or delaying the onset of PCD in the subject.

In the methods of the invention, the subject may be one has been diagnosed with PCD having the c.1167+1262A→G splicing mutation or is suspected of having PCD with the c.1167+1262A→G splicing mutation. In certain embodiments, the subject is an infant or child, e.g., less than 18 years old, e.g., less than 18, 17, 16, 15, 14, 13, 12, 11, 10, 9, 8, 7, 6, 5, 4, 3, 2, or 1 years old.

In some embodiments, an oligonucleotide endosomal compound is administered concurrently or sequentially with a splice switching oligonucleotide. The oligonucleotide endosomal compound can increase the delivery and/or increase the activity of the splice switching oligonucleotide in the cell. The oligonucleotide endosomal compound may be present in the same composition as the oligonucleotide or in a separate composition. Exemplary oligonucleotide endosomal compounds can be found in, for example, U.S. Pat. No. 10,266,823, which is herein incorporated by reference in its entirety. For further example, oligonucleotide endosomal compounds can be compounds of Formula I:

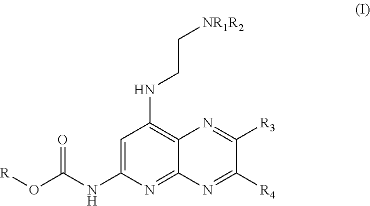

wherein:
R is ethyl or a linking group (preferably ethyl);
R₁ is methyl or a linking group (preferably methyl);
R₂ is methyl;
R₃ and R₄ are each independently H, lower alkyl; lower alkoxy, halo, amino, aryl, or heteroaryl;
or a pharmaceutically acceptable salt thereof.

### Subjects, Pharmaceutical Formulations, and Modes of Administration

Splice switching oligonucleotides (SSOs) according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, rodents, lagomorphs, etc. Human subjects include neonates, infants, juveniles and adults.

In particular embodiments, the present invention provides a pharmaceutical composition comprising a splice switching oligonucleotide of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The splice switching oligonucleotide may be introduced into the cells according to standard transfection methods suitable for the particular target cells. The amount of splice switching oligonucleotides to administer can vary, depending upon the target cell type and number, and the particular splice switching oligonucleotide, and can be determined by those of skill in the art without undue experimentation.

The cell(s) into which the splice switching oligonucleotide is introduced can be of any type. Moreover, the cell can be from any species of origin, as indicated above. The cell(s) can be from airway epithelia or any tissue effected by PCD such as epithelial cells from lung tissue (e.g., nasal, bronchial, tracheal, alveolar (e.g., alveolar type II (ATII)), sweat duct/gland, mammary, intestinal, colon, pancreatic, ocular (e.g., lens and/or retinal), lachrymal duct, otic epithelia, sperm, circulatory endothelium, peritoneal mesothelium, pleural cavity, pericardial cavity, esophageal epithelium, gingival, vaginal, corneal, oral, kidney tubule, ovarian, bronchial, airway gland, mammary gland, sweat gland, salivary gland, gastric, intestinal, uterine, tracheal, fallopian tube, ocular conjunctiva, urethra, pharynx, brain ventricles, bone marrow and blood, muscle, small intestine, large intestine, gall bladder, thyroid follicles, anus, vas deferens, lymph vessel, skin, endometrium, middle ear, epididymis, and/or cervix epithelia.

A further aspect of the invention is a method of administering the splice switching oligonucleotide to subjects. Administration of the splice switching oligonucleotide according to the present invention to a human subject or an animal in need thereof can be by any means known in the art. Optionally, the splice switching oligonucleotide is delivered in a treatment effective or prevention effective dose in a pharmaceutically acceptable carrier.

Dosages of the splice switching oligonucleotide to be administered to a subject depend upon the mode of administration, the disease or condition to be treated and/or prevented, the individual subject's condition, the particular splice switching oligonucleotide, and the nucleic acid to be delivered, and the like, and can be determined in a routine manner.

In particular embodiments, more than one administration (e.g., two, three, four or more administrations) may be employed to achieve the desired level of gene expression over a period of various intervals, e.g., daily, weekly, monthly, yearly, etc.

In particular embodiments, administration can be local or systemic. For example, delivery of SSOs encompasses situations in which a SSO is delivered to a target tissue and the corrected protein is expressed and retained within the target tissue (also referred to as “local distribution” or “local delivery”), and situations in which a SSO is delivered to a target tissue and the corrected protein is expressed and secreted into patient's circulation system (e.g., serum) and systematically distributed and taken up by other tissues (also referred to as “systemic distribution” or “systemic delivery). The delivery can be to airway epithelial cells or any tissue affected by PCD such as epithelial cells from lung, nose, ear, eye, nervous system, and the gastrointestinal and reproductive tract tissues.

Injectables can be prepared in conventional forms, either as liquid solutions or suspensions, solid forms suitable for solution or suspension in liquid prior to injection, or as emulsions. Alternatively, one may administer the splice switching oligonucleotide of the invention in a local manner, for example, in a depot or sustained-release formulation.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal, intrathecal), topical (i.e., both skin and mucosal surfaces, including airway surfaces), intranasal, transdermal, intraarticular, intracranial, intrathecal, cerebrospinal, and inhalation administration, otic administration, ocular administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

The oligonucleotide can alternatively be formulated for nasal, otic, or ocular administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Having described the present invention, the same will be explained in greater detail in the following examples, which are included herein for illustration purposes only, and which are not intended to be limiting to the invention.

### Example 1

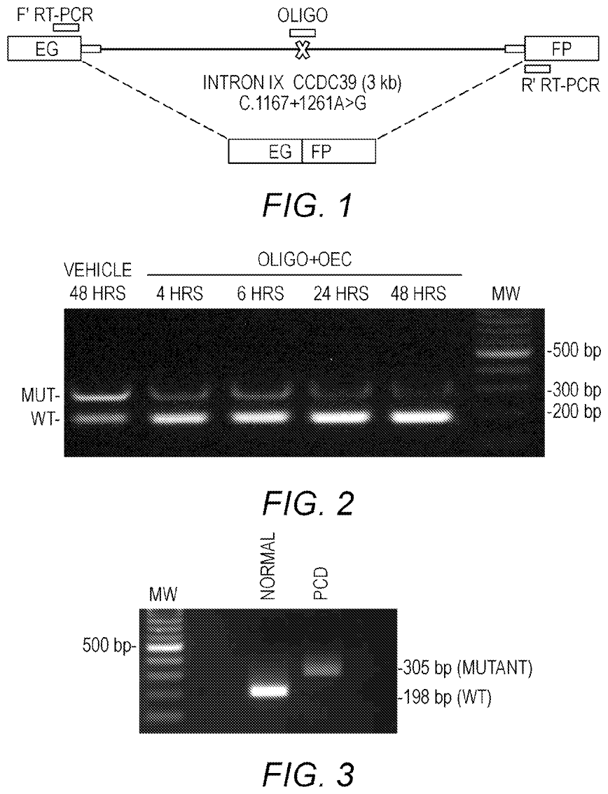

CCDC39 mini-gene. The mini-gene, encoding the EGFP cDNA interrupted by the whole annotated human intron IX from the human CCDC39 gene containing the c.1167+1261A→G splicing mutation (FIG. 1), was synthesized and cloned in the pQCXIP plasmid. The mini-gene plasmid construct was transfected into HEK and HeLa cells. Cells expressing the mini-gene were used to screen sequence-specific splice switching oligonucleotides (SSO).

MOE Splice Switching Oligonucleotide (SSO). A splice switching oligonucleotide was optimized in silico and then synthesized as a 2′-O-methoxyethyl (MOE) modified SSO (all nucleotides) (SEQ ID NO:1). The SSO was administered to HEK cells with vehicle or with an oligonucleotide endosomal compound.

Correction of mRNA expression was analyzed by RT-PCR (FIG. 2) using 2 sets of primers (F′ and R′) at 4, 6, 24, and 48 hours post treatment. Two bands were observed, mutant (˜300 bp) and WT (˜200 bp). PCR bands were sequenced, which confirmed that the mutated mini-gene was corrected into wild type EGFP in cells treated with the MOE oligonucleotide. The data show that the normal processing “splicing out” of intron IX was observed and increased over time post treatment.

### Example 2

PMO splice switching oligonucleotide. A phosphorodiamidate morpholino oligomer (PMO) (all nucleotides) (SEQ ID NO:2) was synthesized in Gene Tools based on the MOE SSO sequence.

The PMO (SEQ ID NO:2) was conjugated to a peptide to produce the SSO-PPMO oligonucleotide. The peptide sequence was RXRRXRRXRRXRXB (SEQ ID NO:3) (Dang et al., Nucleic Acids Res. 49 (11): 6100 (2021)) and covalently linked to PMO CCDC39. The one letter codes for this sequence represent: R=arginine; B=β-alanine; and X=6-aminohexanoic acid.

Induced pluripotent stem cells (iPSCs) were generated from blood cells obtained from a normal and a PCD patient with the CCDC39 c.1167+1261A→G splicing mutation. The cells were lysed, mRNA extracted, and analyzed by RT-PCR for CCDC39 intron IX using PCR primers. Only the mutant form of the mRNA is present in PCD cells (part of intron IX is improperly included) and the normal form of the mRNA is present in normal cells (intron IX is properly omitted) (FIG. 3). The mutant band is larger because the mutation creates a new splicing site allowing for the inclusion of an intronic sequence as a “pseudo” exon, eliciting an aberrant mRNA.

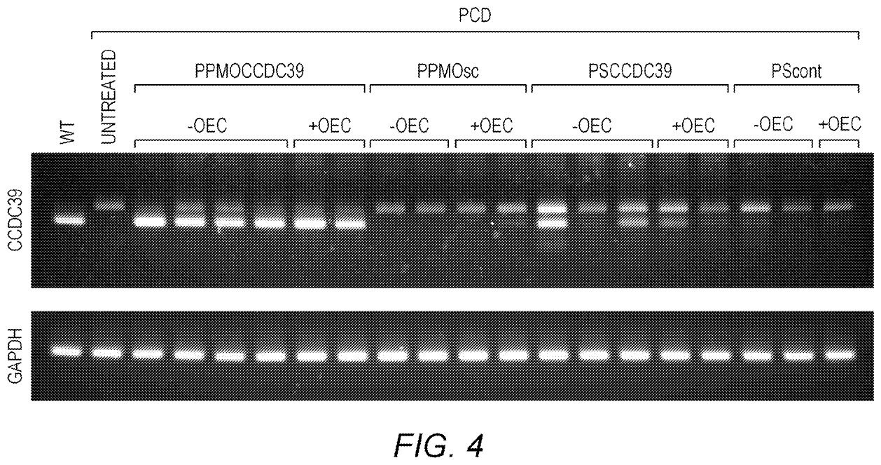

IPSC generated from the PCD patient were tested with the SSOs (1 μM, overnight) with or without the oligonucleotide endosomal compound (10 μM, 1 hour). Both SSOs were tested: the original PS-MOE and the PPMO prototype. Both SSOs showed correction of the splicing mutation 6 hours post treatment and that inclusion of the oligonucleotide endosomal compound may not be necessary (FIG. 4). Controls included iPSC from a normal patient, untreated PCD cells, a PPMO with a scrambled PMO sequence and a non-targeted PS SSO. GAPDH was used as a loading control.

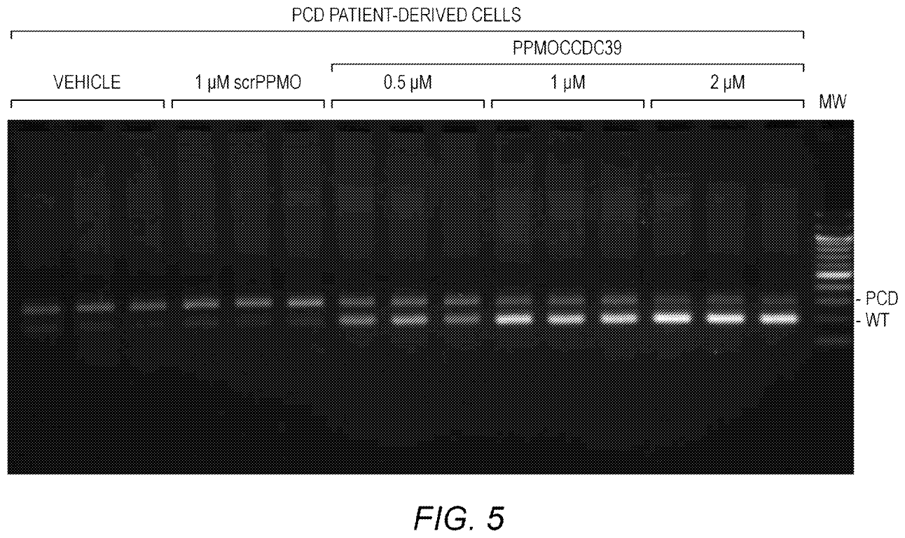

A dose-response study of the SSO-PPMO was performed in iPSC derived from a patient homozygous for the CCDC39 c.1167+1261A>G splicing defect. The SSO/PPMOCCDC39, a scramble PPMO, and vehicle were added to the patient-derived cells at the concentrations indicated in FIG. 5 for 24 h. Cells were analyzed for CCDC39 mRNA splicing/expression by PCR/gel analysis using standard lab procedures. The mRNA was subjected to sequencing indicating that the “corrected” PCR band was indeed the product of normal (wild type) splicing. RNAseq analyses revealed no significant cell toxicity caused by the SSO/PPMOCDC39 at 1 or 2 μM concentrations compared with vehicle or control scramble PPMO.

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A splice switching oligonucleotide for correcting a c.1167+1262A→G mutation in a human CCDC39 gene, wherein the oligonucleotide specifically hybridizes to an mRNA produced from the mutated CCDC39 gene at a site within 100 nucleotides of the mutation.

2. The splice switching oligonucleotide of claim 1, which specifically hybridizes to an mRNA produced from the mutated CCDC39 gene at a site within 25 nucleotides of the mutation.

3. The splice switching oligonucleotide of claim 1, comprising at least 5 consecutive nucleotides of the sequence of SEQ ID NO: 1 or SEQ ID NO:2.

4. The splice switching oligonucleotide of claim 1, wherein the oligonucleotide comprises a sequence at least 70% identical to SEQ ID NO: 1 or SEQ ID NO:2.

5. The splice switching oligonucleotide of claim 4, comprising the nucleotide sequence of SEQ ID NO: 1 or SEQ ID NO:2.

6. (canceled)

7. The splice switching oligonucleotide of claim 1, wherein one or more nucleotides are chemically modified.

8. The splice switching oligonucleotide of claim 7, wherein all of the nucleotides are chemically modified.

9. The splice switching oligonucleotide of claim 7, wherein the oligonucleotide comprises a phosphorodiamidate morpholino backbone.

10. The splice switching oligonucleotide of claim 7, wherein the oligonucleotide comprises one or more 2′-O-methoxyethyl nucleotides.

11. The splice switching oligonucleotide of claim 1, further comprising a peptide conjugated to the oligonucleotide.

12. The splice switching oligonucleotide of claim 11, wherein the peptide sequence is at least 90% identical to RXRRXRRXRRXRXB (SEQ ID NO:3), wherein R is arginine, B is β-alanine, and X is 6-aminohexanoic acid.

13. The splice switching oligonucleotide of claim 12 comprising the peptide sequence of RXRRXRRXRRXRXB (SEQ ID NO:3), wherein R is arginine, B is β-alanine, and X is 6-aminohexanoic acid.

14. A pharmaceutical composition comprising the splice switching oligonucleotide of claim 1 in a pharmaceutically acceptable carrier.

15. The pharmaceutical composition of claim 14, further comprising an oligonucleotide endosomal compound.

16. A method of correcting a c.1167+1262A→G mutation in a CCDC39 gene in a cell, comprising contacting the cell with the splice switching oligonucleotide of claim 1.

17. A method of correcting a c.1167+1262A→G mutation in a pre-mRNA produced from a CCDC39 gene in a subject, comprising administering to the subject an effective amount of the splice switching oligonucleotide of claim 1, thereby correcting the c.1167+1262A→G mutation.

18. A method of treating or delaying the onset of primary ciliary dyskinesia (PCD) in a subject in need thereof, comprising administering to the subject a therapeutically effective amount of the splice switching oligonucleotide of claim 1, thereby treating or delaying the onset of PCD in the subject.

19. The method of claim 17, wherein the subject is a human subject.

20. The method of claim 17, wherein the subject has been diagnosed with PCD.

21. The method of claim 17, further comprising administering an oligonucleotide endosomal compound to the subject.

22. (canceled)
